(19) United States
(12) Patent Application Publication
(10) Pub. No.: US 2010/0145213 A1
(43) Pub. Date: Jun. 10, 2010

(54) LUNG DIAGNOSIS APPARATUS WITH TWO ULTRASOUND MEASUREMENT ZONES

(76) Inventor: Peter Ganshorn, Munnerstadt (DE)

Correspondence Address: EDWIN D. SCHINDLER, FIVE HIRSCH AVENUE, P.O. BOX 966, CORAM, NY, 11727-0966, US

(21) Appl. No.: 12/629,869

(22) Filed: Dec. 2, 2009

(30) Foreign Application Priority Data
Dec. 6, 2008 (DE) 10 2008 060 922.6

(51) Int. Cl. A61B 5/087

(52) U.S. Cl. 600/538

(57) ABSTRACT

A lung diagnosis apparatus for measuring the flow rate and molar mass of the respiratory air of a living organism includes a respiration tube, through which respiratory air can flow. On the outer surface of the respiration tube there are mounted two tube nozzles for which the longitudinal axis, or nozzle axis, extends in an inclined manner relative to the longitudinal axis, or tube axis, of the respiration tube, and in which a first ultrasound transmitter or a first ultrasound receiver is fixed. An electronic module, which actuates the ultrasound transmitter and the ultrasound receiver also evaluates their ultra-sound signals. The nozzle axis for each tube nozzle pass through a reflection point on the inner surface of the respiration tube. A second ultrasound transmitter and a second ultra-sound receiver are on the outer surface of the respiration tube coaxially with one another and approximately orthogonal to the respiration tube axis with the sound radiated by the second ultrasound transmitter being oriented to the reflection point.

The invention relates to a lung diagnosis apparatus for measuring the flow rate, and the molar mass of the respiratory air of a living organism, comprising a respiration tube, through which the respiratory air flows, and on the outer surface of which there are mounted two tube nozzles, of which the longitudinal axis—the nozzle axis—extends inclined with respect to the longitudinal axis of the respiration tube, the tube axis, and in which, in each case, a first ultrasound transmitter or a first ultrasound receiver is fixed, and an electronic module, which actuates the ultrasound transmitter and the ultrasound receiver and evaluates their signals.

For the diagnosis of different lung Illnesses, it is necessary to determine the flow rate, and the molar mass of the respiratory air. Various methods and various apparatus are known for this. In the prior art, European Patent EP 0 653 919, Hamoncourt, describes a measurement zone, through which respiratory air flows and on which an ultrasonic transmitter-receiver pair is arranged obliquely to the longitudinal axis of a measurement tube. With these two cells, the travel time of an ultrasonic pulse through the flowing respiratory gas is measured and, from this, the flow rate is calculated. In addition, one temperature sensor in each case is disposed at the entrance to and outlet from the measurement zone. The measurement value for the gas temperature and the value of the respective sound velocity—which can be derived from the travel-time measurements—permits the calculation of the respective molar mass.

A significant disadvantage of this measurement process is that the principle excludes a further increase of the measurement accuracy or the molar mass, since the exact temperature of the gas in the measurement zone is not known, but it must be assumed as an average value between the values measured at the inlet and outlet.

Further, even more serious disadvantages are caused by the fact that the ultrasonic transmitter and the ultrasonic receiver must be installed in pipe nozzles that are arranged obliquely on the outer surface. These tube nozzles mounted obliquely to the respiration tube could only be dispensed with if the ultrasonic transmitter and ultrasonic receiver were known, which would have a trapezoidal cross-section in the longitudinal section, so that their sound outlet surface or their sound inlet surface would adapt to the shape of the tube interior. Since, however, such transmitters and receivers are unknown in the prior art, both tube nozzles, which are mounted obliquely on the respiration tube, must be regarded as currently the lesser evil.

The advantage of the nozzle is that the ultrasonic transmitter and the ultrasonic receiver, despite their included arrangement with respect to the longitudinal axis of the respiration tube do not project into the respiration tube. Otherwise they would hinder the flow of the respiratory air there and thereby falsify the measurements of the flow rate.

The disadvantages of the tube nozzle are that the respiratory air that has penetrated therein undergoes turbulence, so that the uniform flow of the breath is interrupted. Thus the velocity of the moist respiratory air is also reduced, as a result of which it cools. down. A further cooling results from the disclosed form of the nozzle.

For the measurement of the molar mass in the temperature range between about 30° and 35°, a change of temperature by 1° effects an average change of the measurement value of the molar mass of the order of about 10%. The example illustrates the considerable dimension of this error.

A further error occurs due to the change of CO₂ concentration of the respiratory air in the tube nozzle.

Although, to an approximation, this error can be theoretically compensated, the measurement accuracy remains very restricted.

It is also known to arrange close-meshed grilles between the tube nozzle and the interior of the respiration tube, which reduce the penetration of respiratory air into the tube nozzle, however do not restrict the ultrasound on the measurement zone too much. However, with this arrangement, turbulence of the respiratory air in the tube nozzle always remains, which limits the measurement accuracy.

Against this background it is the object of the invention to provide an arrangement for measuring the flow rate and the molar mass of the respiratory air, which permits a significantly higher measurement accuracy compared to the prior art without significantly increasing the outlay for the apparatus.

As a solution, the invention teaches that the two nozzle axes each pass through a reflection point on the inner surface of the respiration tube and a second ultrasound transmitter and a second ultrasound receiver on the outer surface of the respiration tube are arranged coaxially with respect to one another and approximately orthogonally to the tube axis, and the sound radiated from the second ultrasound transmitter is oriented to a reflection point The invention is also distinguished from the prior art by two essential features: First, it is a second measurement zone that is arranged transversely to the respiratory air, so that the molar mass can be computed without the errors from estimating the temperature and without the errors due to the air turbulence within the tube nozzle. The second, characterizing feature is a significant extension of the measurement zone for determining the flow rate and increasing the measurement accuracy for this value, since—with an equal angle between the nozzle axis and the tube axis—due to the reflection of the ultrasound pulses in the respiration tube, the measurement zone is twice as long.

By virtue of the reflection of the ultrasound pulses in the second measurement zone for determining the molar mass, too, the length of this measurement zone, at twice the diameter of the respiration tube, is in the majority of cases longer than in the prior art with only a single ultrasound measurement cell pairs.

It is possible that the first measurement zone for determining the flow rate is not only reflected at a reflection point, but is multiply reflected. By this means, a further elongation of the measurement zone is possible. A restriction of the number of reflection points results from the fact that, with an overlarge difference between the lowest and highest velocity of the respiratory air, the multiply reflected ultrasound signal is deflected to the extent that, with a very high air velocity, it can no longer be perfectly received by the ultrasound receiver.

In the interest of the simplest possible arrangement, too, the invention therefore prefers an arrangement with only a single reflection point. This reflection point is then also used for the second ultrasound measurement cell pair for measuring the molar mass. By this means, it is ensured that the acquisition of the flow velocity and the acquisition of the molar mass always take place at exactly the same point in time and at exactly the same position. Thus—in the interests of the object of the invention—errors are avoided, which arise as a result of different points in time or different positions of the measurement of the two parameters.

In the simplest case, the respiration tube has a circular cross-section. The surface that surrounds the reflection point is then—like the other regions of the tube—a cylindrical segment. Ultrasound waves that are incident on such a cylindrical segment are, seen in the longitudinal direction of the respiration tube, emitted at the same angle as they are incident, Since the surface around the reflection point is slightly curved transversely to the longitudinal axis, that is to say in the radial direction, the incident ultrasound waves are thereby reflected in a somewhat different direction in each case, dependent on the point of incidence. In this manner, a certain focusing of the reflected sound waves takes place,

This effect does not occur when the surface around the reflection point is designed as a plane. Then the angle between the incident and reflected ultrasound is always identical and very effectively predictable, This development of the surface around the reflection point is in particular expedient if the ultrasound transmitter emits the sound waves with relatively strong focusing.

For other ultrasound transmitters that distribute the sound over a larger emission angle, a focusing of the sound waves in the reflection point would be expedient. With this design of ultrasound transmitters, it is an obvious step to design the surface surrounding the reflection point as a calotte, on the inner surface of which a tangent is oriented in all points perpendicular to the angle bisector between the incidence direction of the ultrasound and its emission direction. In this embodiment, the sound waves—in a similar way to, e.g., light rays through the reflector of a headlamp—are brought together at a particular point, in this case expediently in the ultrasound receiver. By this means, the ultrasound transmitter can operate with a somewhat lower power and/or permit a somewhat reduced focusing of the emitted sound waves.

If the surface that surrounds the reflection point has a particular shape deviating from the remaining shape of the respiration tube, the invention proposes that the transition between the inner surface of the respiration tube are the surface surrounding the reflection point is continuous, so that the flow of the respiratory air is affected as little as possible.

For the special case that this surface is a plane, it is proposed that the profile of the respiration tube in this area is a plane over almost the entire length, in this subvarlant, too, the transition from the plane to the rest of the region of the inner surface of the respiration tube should be continuous in order to avoid the formation of excess turbulence and for the respiratory air to flow as laminar as possible through the measurement zone.

A further increase of the measurement accuracy can be achieved in that the ultrasound transmitters are also usable as receivers and the ultrasound receivers are also usable as transmitters.

By this means, a measurement in alternating directions is possible, so that the errors from directionally dependent influences can be compensated.

As a further improvement, the function of the second ultrasonic transmitter and the second ultrasonic transmitter arranged coaxially to it can be combined in a single module, which operates alternately as transmitter and as receiver. In this configuration, a lung diagnosis apparatus according to the invention is equipped with only three ultrasound elements, of which each operates both as a transmitter and as a receiver.

As already mentioned above, it is expedient that both tube nozzles are separated from the interior space of the respiration tube by means of a grille in each case that mostly permits the ultrasound to pass though and mostly holds back the respiratory air. By this means, inter alia, the formation of disturbing turbulence within the tube nozzle is at least highly suppressed. The desirable hygienic standard in the region of the tube nozzle can also be realized with very much lower outlay. Nevertheless, a transmissibility for ultrasound is to be ensured; therefore, for example, a closed foil would falsify the measurement due to its oscillation behaviour, which would additionally change significantly with time.

To design the transmissibility for ultrasound through the grille to be as efficient as possible, it is expedient to choose a very thin material in which the regular series of openings necessary for a grille are introduced. So that this material is not set into strong oscillations during the air flow, it should be supported by at least one crosspiece. Here, it should be preferred that the crosspiece has an elongated profile whose longitudinal axis extends approximately parallel to the support axis. By this means, the crosspiece directs the least possible surface area to the ultrasound and at the same time provides the grille with the best possible stabilization.

In a further variant of a lung diagnosis apparatus according to the invention, a two-part construction of the respiration tube is proposed: An inner tube is inserted into an outer tube.

The inner tube guides the respiratory air, is interchangeable by the user and contains all the reflection points. On an approximately axially extending line, it comprises three openings, of which at least the first and the last are closed by means of a grille, which is hardly transmissible for the respiratory air, but mostly allows the ultrasonic waves to pass through:

Furthermore, the inner tube comprises a reflection point on the inner side opposite the central window. in the simplest embodiment, this reflection point and its direct surroundings are only a portion of the uniformly passing-through, inner wall surface of the respiration tube. Alternatively, the surrounding of the reflection point can also be designed as a plane or as a calotte or another shape particularly suitable for the reflection of ultrasound waves and/or of a particularly suitable material.

The outer tube comprises all ultrasound elements and other measurement devices and is permanently integrated into the lung diagnosis apparatus. Opposite the central opening of the inner tube there are arranged the second ultrasound transmitter and the second ultrasound receiver oriented coaxially thereto, or only a single ultrasound element that operates alternately as a transmitter and receiver.

Opposite the first of the two outer openings of the inner tube, a pipe nozzle is formed in the outer tube and includes the first ultrasound transmitter and, opposite the second of the two outer openings of the inner tube, the second tube nozzle is integrally formed, which receives the first ultrasound receiver, which, together with the first ultrasound transmitter, forms the first measurement zone. Alternatively, in both tube nozzles, ultrasound elements are incorporated, which operate alternately as transmitter and as receiver.

The first ultrasound transmitter and the first ultrasound receiver use the same reflection point as the second ultrasound transmitter and the second ultrasound receiver, only with the difference that the sound waves of the first measurement zone travel obliquely to the respiratory flow.

This arrangement offers numerous advantages. The outer tube for receiving the ultrasound receiver and transmitter is relatively easy to produce, since it does not contain branched cavities. After the removal of the inner tube for air guidance, the two tube nozzles are very readily inspectable and easy to clean. The fastening lying therebetween for the ultrasound elements of the second measurement zone can also be readily inspected and easily cleaned.

This arrangement is very advantageous with respect to the prior art, in particular when the inner tube with the grille openings is used as a disposable part for once-only use.

In a further embodiment further simplifications can be made during insertion of the inner tube into the outer tube. The outer tube is reduced to a hollow cylindrical segment that contains the nozzle and a holder for three ultrasound elements. Into this hollow cylindrical segment, the inner tube is inserted and can be pressed, e.g. by means of a band, onto the outer-tube segment.

During insertion, this band can be brought to a diameter that is greater than that of the respiration tube, so that the respiration tube can be easily pushed in. When the respiration tube has been brought to the correct position, in which the grille-covered openings stand opposite the two tube nozzles, the band can be tightened again, e.g. by means of a lever. Of course, any other tensioning devices are applicable.

The production of the outer tube is also simplified. Unlike in the prior art, all grille-covered openings face only one side, so that they can be injection moulded with a common die.

After the removal of the inner tube, the moulded part with the two pipe nozzles and the three ultrasound elements can be easily cleaned and disinfected.

In practice, the respiration tube can be chiefly manufactured as a plastic injection molding. In the prior art, openings would have to be introduced into this plastic part on two opposite sides and closed with the air-blocking grille. For this purpose, corresponding tools on both sides of the mold would be necessary. The respiration tube of a lung diagnosis apparatus according to the invention with only one reflection point—or an odd number of reflection points—has, on the other hand, openings on only one side, which are to be produced with a respiratory air protection grille.

By this means, the same tool can be used twice for this respiratory air protection grille, which significantly simplifies the manufacturing process.

Or all windows are combined into a common, somewhat larger window, for which only one tool is required.

Since the respiration tube with its complex shape is a relatively expensive part in the entire arrangement and since the production number on this market is very low relative to other plastic injection mouldings, the mould makes up a very high proportion of the total costs, it is therefore expedient to keep the form as small as possible and thereby design it as inexpensively as possible, and on the other hand to increase the number of units produced in this form.

For this, the invention proposes as an embodiment also the normally unusual concept of dividing the respiration tube into two identical parts, of which the separating face extends between the second ultrasound transmitter and the second ultrasound receiver. The respiration tube is thus divided approximately in its centre. The identical shape between the two halves presupposes that the inlet of the respiratory air at the mouth is identical to the outlet at the opposite part of the respiration tube. Furthermore, the connecting surface must be designed such that it is divided by a radially extending axis into two halves that are complementary to one another. In these two sections, e.g. mutually corresponding detent hooks and detent lugs can be provided, so that two identical halves of the respiration tube, which are pivoted by 180° with respect to one another, can be snapped into one another at the connecting point.

This embodiment naturally presupposes that the mechanical reception of the ultrasound element in one half of the respiration tube of the ultrasound element in one half of the respiration tube is also suitable for the mechanical reception of a, then, equally sized ultrasound element in the other half of the respiration tube.

If these two mutually identical parts of the respiration tube have to be produced with only low precision, and if no inner tube is inserted that contains the reflection point, the achieved tolerance of the surface around the reflection point can be low. For this case, the invention proposes to introduce a receptacle in the joining surface of the two respiration tube halves, into which a third part of the reflection point together with the surrounding surface is plugged.

In the application of a lung diagnosis apparatus according to the invention it is particularly expedient to use the first ultrasound measurement zone, consisting of the first ultrasound transmitter and the first ultrasound receiver, whose measurement signal crosses the direction of the respiratory air at an oblique angle, for measuring the flow velocity.

On the other hand, for measuring the molar mass, the second ultrasound measurement zone should be used, which is not only impaired by the measurement error of the nozzles, but whose achievable measurement accuracies are a multiple higher than when this value is calculated from the measurements of the first ultrasound measurement zone, it only being possible to inadequately compensate the temperature gradients, the turbulence effects and the changes of the CO₂ concentration.

Further details and features of the invention are explained below in greater detail with reference to an example. The illustrated example is not intended to restrict the invention, but only to explain it. In schematic view,

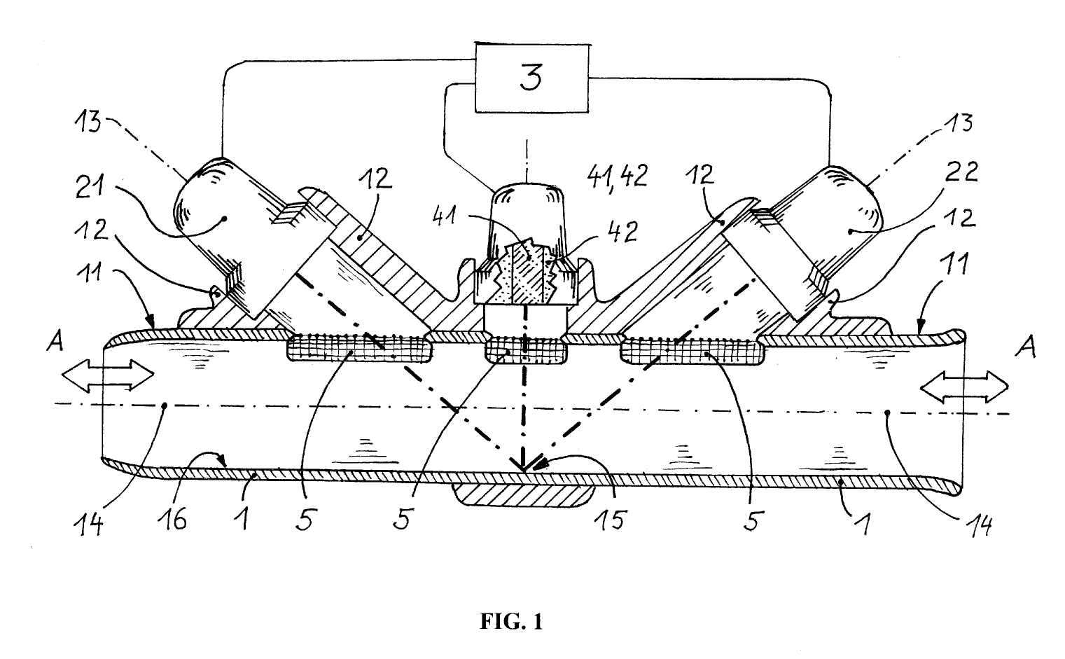

FIG. 1 shows a longitudinal section through a lung diagnosis apparatus according to the invention

In FIG. 1, the principle construction elements of a lung diagnosis apparatus according to invention are shown in cross-sectional view through their length. Conspicuous is the elongated respiration tube 1, through which the respiratory air A of the living organism to be diagnosed streams. In the illustrated embodiment, a total of three grilles 5 are inset into the outer surface 11 of the respiration tube 1, which separate the interior space of the respiration tube A from interior space of the two tube nozzles 12.

At the ends of these two tube nozzles 12, which are arranged at the left and right, are mounted the first ultrasound transmitter 21 and the first ultrasound receiver 22. The first ultrasound transmitter 21 radiates ultrasound pulses in the direction of the longitudinal axis of the pipe nozzle 12 of the so-called “nozzle axis 13”. The nozzle axis 13 is oriented such that it meets the inner surface 16 of the respiration tube 1 in the reflection point 15. Thereby the ultrasound pulses emitted by the ultrasound transmitter 21 also meet the reflection point 15. The surface actually required around the reflection point 15 is dimensioned depending on the focusing of the ultrasound pulse. There, the ultrasound pulses are reflected and guided by the second tube nozzle 12 to the first ultrasound receiver 22.

The electronic module 3—symbolized just by a block here—actuates the ultrasonic transmitter 21 and determines the travel time change—effected by the current of respiratory air—of the ultrasound pulses received in the ultrasound receiver 22 and from this calculates the flow velocity of the respiratory air A.

In FIG. 1, it very quickly becomes clear that, in the embodiment shown here, the two pipe nozzles 12, together with the holder for the second ultrasound measurement zone 41, 42, lie as a separate module on the inner tube of the respiration tube 1, and are fastened at the opposite side only by means of a narrow clip.

The second ultrasound measurement zone 41, 42 consists of the second ultrasound transmitter 41 and the second ultrasound receiver 42, which are arranged coaxially with one another. In this embodiment, the second ultrasound transmitter 41—shown here schematically as a block—is surrounded by the approximately annular second ultrasound receiver 42.

In FIG. 1 it can be clearly seen that the second ultrasound measurement zone 41, 42 uses the same reflection point 15 as the first ultrasound measurement zone 21, 22. That has the advantage—compared to the prior art—that the flow velocity is measured at exactly the same point as the molar mass, that is to say the same molecules are actually registered by the two measurements. It is readily evident that the measurement accuracy is thereby further increased.

The second ultrasound measurement zone 41, 42 comprising the second ultrasound transmitter 41 and the second ultrasound receiver 42, is also actuated and evaluated via the electronic module 3.

In FIG. 1 it is not shown that the electronic module 3 in practice can also be set up for output of the measurement values. Even an interpretation of the determined measurement values is possible in the electronic module.

### LIST OF REFERENCE CHARACTERS

A Respiratory air of a living organism

1 Respiration tube through which respiratory air flows

11 Outer surface of the respiration tube 1

12 Tube nozzle on outer surface 11

13 Nozzle axis, longitudinal axis of a tube nozzle 12

14 Tube axis, longitudinal axis of the respiration tube

15 Reflection point on the inner surface 18 of the respiration tube 1

16 Inner surface of the respiration tube 1

21 First ultrasonic transmitter in a tube nozzle 12

22 First ultrasonic receiver in a tube nozzle 12

3 Electronic module, actuates the ultrasonic transmitter 21 and the ultrasonic receiver 22 and evaluates their signals

41 Second ultrasound transmitter on outer surface 11

42 Second ultrasound receiver on outer surface 11, arranged coaxially to the second ultrasound transmitter 41

5 Grille between the inner space of the respiration tube 1 and one tube nozzle 12 in each case

What is claimed is:

1. A lung diagnosis apparatus for measuring flow rate and molar mass of respiratory air of a living organism, comprising:
a respiration tube though which respiratory air flows having an outer surface with a first tube nozzle and a second tube nozzle, each said tube nozzle having a longitudinal axis defining a first nozzle axis and a second nozzle axis, respectively, extending in an inclined manner relative to a longitudinal axis of said respiration tube, said first nozzle axis and said second nozzle axis each passing through a reflection point on an inner surface of said respiration tube;
a first ultrasound transmitter fixed in said first tube nozzle;
a second ultrasound received fixed in said second tube nozzle;
an electronic module for actuating said first ultrasound transmitter and said first ultrasound receiver, said electronic module further evaluates signals of said first ultra-sound transmitter and said first ultrasound receiver;
a second ultrasound transmitter on the outer surface of said respiration tube; and,
a second ultrasound receiver on the outer surface of said respiration tube, said second ultrasound transmitter and said ultrasound receiver being arranged coaxially with one another and approximately orthogonally to the longitudinal axis of said respiration tube with sound radiated by said second ultrasound transmitter being oriented to the reflection point on the inner surface of said respiration tube.

2. The lung diagnosis apparatus for measuring flow rate and molar mass of respiratory air of a living organism according to claim 1, wherein said first nozzle axis and said second nozzle axis extend through only a single reflection point, said single reflection point being said reflection point on the inner surface of said respiration tube.

3. The lung diagnosis apparatus for measuring flow rate and molar mass of respiratory air of a living organism according to claim 1, wherein said reflection point is surrounded by a surface shaped as a plane oriented perpendicularly to an angle-bisector between a direction of incidence of ultrasound and its emission direction.

4. The lung diagnosis apparatus for measuring flow rate and molar mass of respiratory air of a living organism according claim 3, wherein a transition between the plane and the inner surface of said respiration tube is continuous.

5. The lung diagnosis apparatus for measuring flow rate and molar mass of respiratory air of a living organism according to claim 1, wherein said reflection point is surrounded by a surface shaped as a calotte, an inner surface thereof having a tangent in all points oriented perpendicularly to an angle-bisector between a direction of incidence of ultrasound and its emission direction.

6. The lung diagnosis apparatus for measuring flow rate and molar mass of respiratory air of a living organism according claim 5, wherein a transition between the calotte and the inner surface of said respiration tube is continuous.

7. The lung diagnosis apparatus for measuring flow rate and molar mass of respiratory air of a living organism according to claim 1, wherein at least one of said first ultrasound transmitter and said second ultrasound transmitter is also an ultrasound receiver.

8. The lung diagnosis apparatus for measuring flow rate and molar mass of respiratory air of a living organism according to claim 1, wherein at least one of said first ultrasound receiver and said second ultrasound receiver is also an ultrasound transmitter.

9. The lung diagnosis apparatus for measuring flow rate and molar mass of respiratory air of a living organism according to claim 1, wherein said second ultrasound transmitter and said ultrasound receiver is a single module operating alternately as an ultrasound transmitter and an ultrasound receiver.

10. The lung diagnosis apparatus for measuring flow rate and molar mass of respiratory air of a living organism according to claim 1, comprising a first grille and a second grille with said first tube nozzle separated from an interior space of said respiration tube by said first grille and said second tube nozzle separated from the interior space of said respiration tube by said second grille, said first grille and said second grille each substantially permitting passage therethrough of ultrasound, while substantially preventing passage therethrough of respiratory air.

11. The lung diagnosis apparatus for measuring flow rate and molar mass of respiratory air of a living organism according to claim 10, wherein said first grille and said second grille are supported by a first crosspiece and a second crosspiece, respectively, said first crosspiece and said second crosspiece each have an elongated profile and a longitudinal axis extending substantially parallel to a supporting axis.

12. The lung diagnosis apparatus for measuring flow rate and molar mass of respiratory air of a living organism according to claim 1, wherein said respiratory tube includes an inner tube and an outer tube.

13. The lung diagnosis apparatus for measuring flow rate and molar mass of respiratory air of a living organism according to claim 12, wherein said inner tube of said respiratory tube includes at least a first opening and a second opening each closed by a first grille and a second grille, respectively, said first grille and said second grille each substantially permitting passage therethrough of ultrasound, while substantially preventing passage therethrough of respiratory air.

14. The lung diagnosis apparatus for measuring flow rate and molar mass of respiratory air of a living organism according to claim 12, wherein said inner tube of said respiratory tube includes the reflection point on the inner surface of said respiration tube.

15. The lung diagnosis apparatus for measuring flow rate and molar mass of respiratory air of a living organism according to claim 12, wherein said outer tube of said respiratory tube supports said second ultrasound transmitter and second ultrasound receiver opposite a central opening of said inner tube.

16. The lung diagnosis apparatus for measuring flow rate and molar mass of respiratory air of a living organism according to claim 12, wherein said inner tube of said respiratory tube includes a single grille through which ultrasound waves of said first ultrasound transmitter, said second ultrasound transmitter, said first ultrasound receiver and said second ultrasound receiver pass.

17. The lung diagnosis apparatus for measuring flow rate and molar mass of respiratory air of a living organism according to claim 1, wherein said respiratory tube includes a first portion and a second portion with a separating surface extending transversely to the longitudinal axis of said respiratory tube and through said second ultra-sound transmitter and said second ultrasound receiver.

18. The lung diagnosis apparatus for measuring flow and molar mass of respiratory air of a living organism according to claim 17, wherein said reflection point on the inner surface of said respiration tube is surrounded by a surface as a third portion between said separating surface separating said first portion and said second portion of said respiration tube.
